(19) United States
(12) Patent Application Publication
(10) Pub. No.: US 2002/0074002 A1
(43) Pub. Date: Jun. 20, 2002

(54) Detecting device for correct endotracheal intubation

(76) Inventors: Chin Sheng Tung, Tainai City (TW); Lim Shen Lee, Tainai City (TW); Jui Hung Hung, Tainai City (TW)

Correspondence Address: Charles E. Baxley, 59 John Street-5th Floor, New York, NY, 10038, US

(21) Appl. No.: 09/738,102

(22) Filed: Dec. 18, 2000

(51) Int. Cl. A61N 5/00; A61F 11/00; A62B 9/06; A61M 16/00

(52) U.S. Cl. 128/207.14; 606/108

(57) ABSTRACT

A detecting device for correct endotracheal intubation includes a tube to be engaged into a trachea of a patient, a signal emitting device for engaging into the trachea of the patient and for emitting a signal, and a sensor for sensing the signal from the signal emitting device to detect the correct engagement of the tube in the trachea of the patient. The signal emitting device may be attached to the tube for engaging into the trachea together with the tube. A control device may treat the signal and may indicate the correct engagement of the tube in the trachea.

### BACKGROUND OF THE INVENTION

1. Field of the Invention

The present invention relates to a detecting device, and more particularly to a detecting device for correct endotracheal intubation.

2. Description of the Prior Art

As shown in FIGS. 8 and 9, for general anaesthesia operation or the like, such as for surgery purposes, a tube 10, such as an endotracheal tube 10 is required to be engaged into the trachea 70 of a patient. An air envelope 11 or the like may be attached to the tube 10, and an air pump 12 or an air nozzle or the like may be coupled to the air envelope 11 to inflate the air envelope 11 and to facilitate the insertion of the tube 10 into the trachea or for facilitating the endotracheal intubation. As shown in FIG. 9, after the tube 10 is correctly engaged into the trachea, an air nozzle or an air pump 16 or the like may be coupled to the tube 10 via a coupler 14 for pumping air into the trachea and the lungs and facilitating the breathing of the patients.

The correct engagement of the tube 10 into the trachea 70 or the correctness of endotracheal intubation is important to the patient. The wrongly engagement of the tube 10 into the esophagus 80 may cause the so-called accidental and unrecognized esophageal intubations and may result in disastrous sequelae. For example, the patients may be suffocated, the heart beat may be stopped, the nervus system may be damaged, etc. A capnography or a carbon dioxide detecting facility 17 may be coupled to the tube 10 via a coupler 14 for detecting the expired carbon dioxide or for detecting the correctness of the endotracheal intubation. However, the carbon dioxide detecting facilities 17 are expensive and bulky and thus may be used in operating rooms only. The facilities may not be easily carried outdoors and thus may not be used for emergency or intensive care or other resuscitation purposes.

The present invention has arisen to mitigate and/or obviate the afore-described disadvantages of the conventional detecting devices for correctness of the endotracheal intubation.

### SUMMARY OF THE INVENTION

The primary objective of the present invention is to provide a detecting device for correct endotracheal intubation which may be used for easily detecting the correct engagement of the tube into the trachea and for preventing the tube from being engaged into the esophagus of the patients.

The other objective of the present invention is to provide a detecting device for correct endotracheal intubation which may be easily carried outdoors.

In accordance with one aspect of the invention, there is provided a detecting device for correct endotracheal intubation comprising a tube to be engaged into a trachea of a patient, a signal emitting device for engaging into the trachea of the patient and for emitting a signal, and means for sensing the signal from the signal emitting device to detect the engagement of the signal emitting device in the trachea of the patient and thus to detect the correct engagement of the tube in the trachea of the patient.

The signal emitting device is preferably attached to the tube for engaging into the trachea of the patient together with the tube and is preferably a magnet or an electric device or a light device for emitting a magnetic signal or an electric or wave signal or a light signal.

The sensing means includes a sensor for sensing the signal from the signal emitting device in order to detect or to locate whether the signal emitting device and the tube are correctly engaged in the trachea of the patient or not, and further includes means for treating the signal from the sensor.

A device may further be provided for indicating the correct engagement of the tube in the trachea of the patient, and may include a lamp for generating a light signal, or an alarm device for generating an alarm signal or a meter for indicating a distance between the sensing means and the signal emitting device.

Further objectives and advantages of the present invention will become apparent from a careful reading of a detailed description provided hereinbelow, with appropriate reference to accompanying drawings.

### BRIEF DESCRIPTION OF THE DRAWINGS

FIG. 1 is an exploded view of a detecting device for correct endotracheal intubation in accordance with the present invention;

FIG. 2 is a partial cross sectional view of a patient for illustrating the engagement of the tube into the trachea;

FIG. 3 is a cross sectional view taken along lines 3-3 of FIG. 1;

FIG. 4 is a plane schematic view illustrating the attachment of the sensor to the patient;

FIG. 5 is an exploded view illustrating the other application of the detecting device;

FIG. 6 is a partial cross sectional view of the patient similar to FIG. 2, for illustrating the engagement of the tube of FIG. 5 into the trachea;

FIG. 7 is a block diagram showing the electric members for the detecting device for correct endotracheal intubation;

FIG. 8 is a perspective view illustrating a typical endotracheal tube to be engaged into the patient; and

FIG. 9 is a partial cross sectional view of the patient, for illustrating the engagement of the typical endotracheal tube into the trachea of the patient.

### DETAILED DESCRIPTION OF THE PREFERRED EMBODIMENT

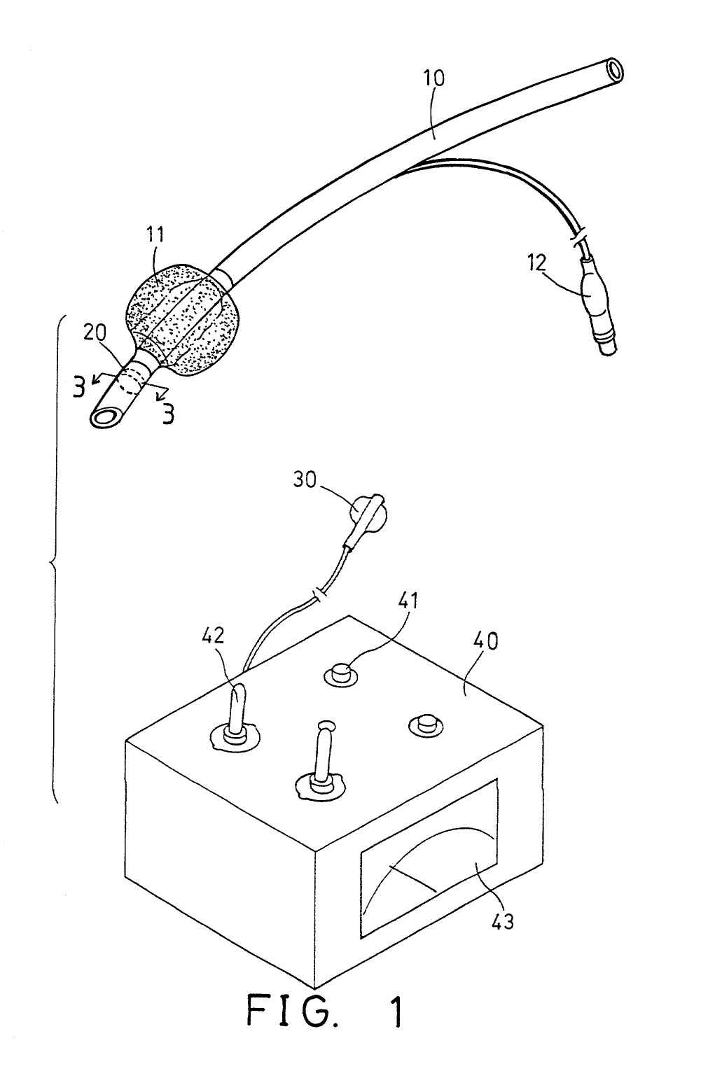

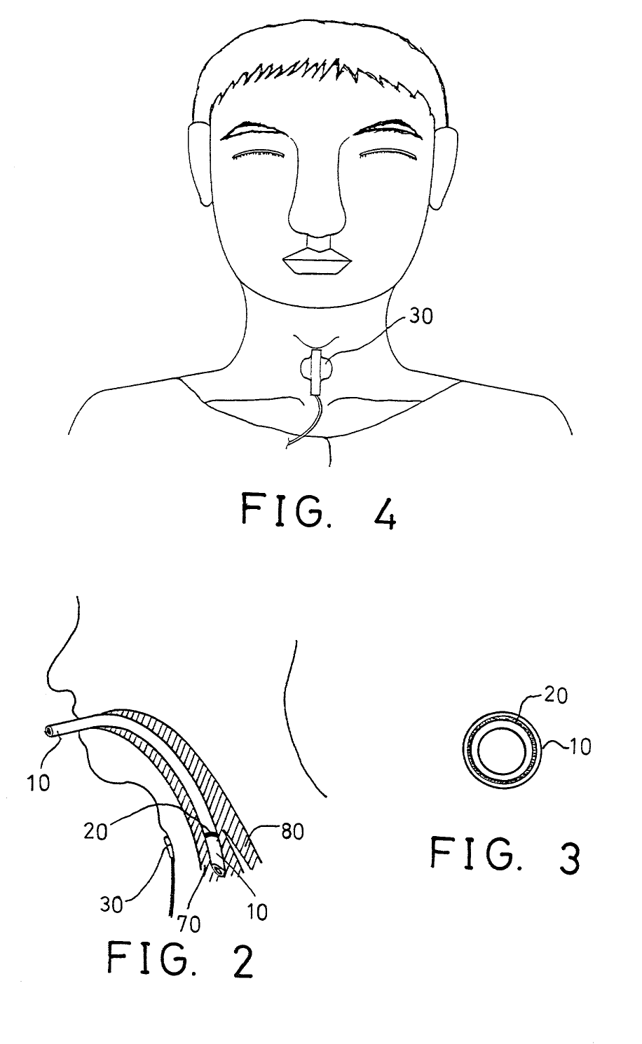

Referring to the drawings, and initially to FIGS. 1-4, a detecting device for correct endotracheal intubation in accordance with the present invention comprises a tube 10, such as an endotracheal tube 10 for engaging into the trachea 70 of a patient, an air envelope 11 or the like may also be attached to the tube 10, and an air pump 12 or an air nozzle or the like may also be coupled to the air envelope 11 to inflate the air envelope 11 and to facilitate the insertion of the tube 10 into the trachea or for facilitating the endotracheal intubation. After the tube 10 is correctly engaged into the trachea, a typical air nozzle or a typical air pump or the like may be coupled to the tube 10 for pumping air into the trachea and the lungs and facilitating the breathing of the patients. The above described structure is typical. The present invention is to provide a detecting device for detecting the correct engagement of the tube 10 into the trachea 70 or the correctness of endotracheal intubation, and for preventing the tube 10 from being wrongly engaged into the esophagus 80 of the patient.

The present invention is to provide a signal emitting device 20 for engaging into the trachea 70 of the patient, and a sensor 30 for detecting the signal emitted from the signal emitting device 20 in order to detect the position of the signal emitting device 20 and thus the tube 10 in the trachea 70 or in the esophagus 80. The signal emitting device 20 may be a magnetic member for generating a magnetic signal, or an electric facility for generating an electric signal or a wave signal, or the like, or a light emitting device for generating a light signal. A protective covering or film or the like may be attached onto the signal emitting device 20 for protecting the signal emitting device 20 and for preventing the signal emitting device 20 from being wetted or damaged by fluid or the like. The sensor 30 may be an integrated circuit or the like for detecting the magnetic or electric or wave or light signals. As shown in FIGS. 1-3, the signal emitting device 20 may be attached to or engaged in the tube 10, particularly to the end portion of the tube 10 that is to be engaged into the trachea 70 of the patient. The sensor 30 may be attached to the throat portion of the patient (FIG. 4) for detecting the signal emitted from the signal emitting device 20 and for detecting and locating the position of the signal emitting device 20 and thus the tube 10 in the trachea 70 or in the esophagus 80.

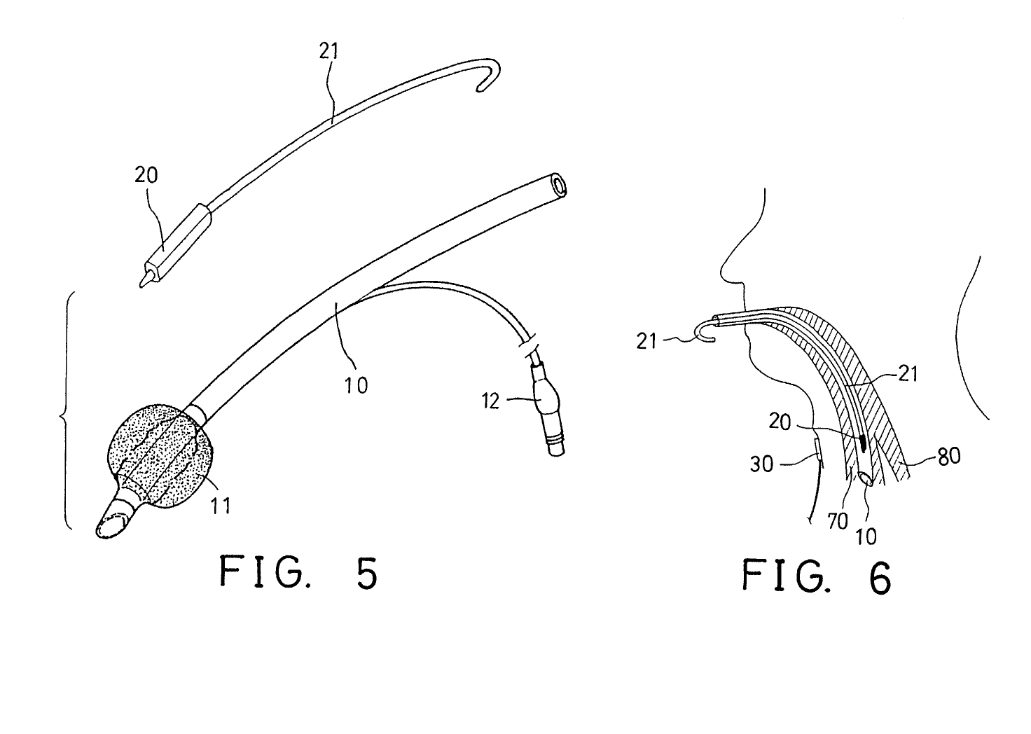

Referring next to FIGS. 5 and 6, alternatively, the signal emitting device 20 may also be attached onto an elongate member 21 and may also be engaged into the tube 10 and thus into the trachea 70 or the esophagus 80, such that the signals generated by the signal emitting device 20 may also be used to locate the trachea 70 or the esophagus 80.

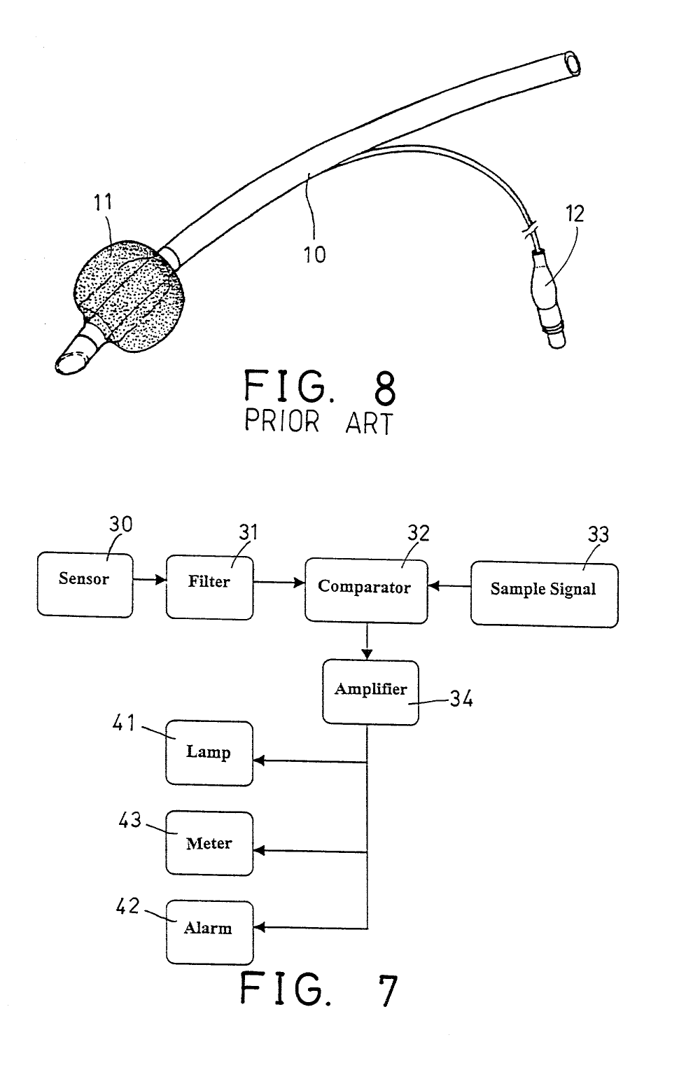

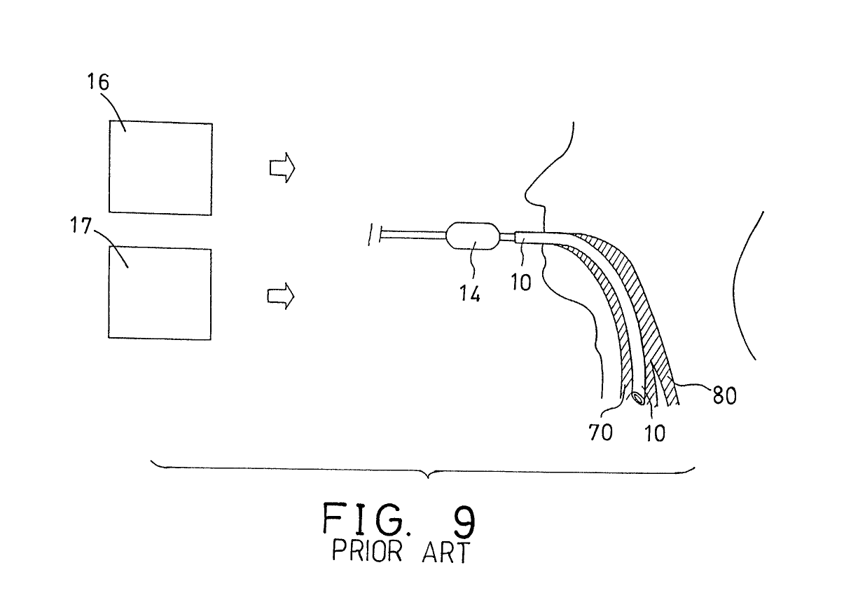

Referring again to FIG. 1, a control device 40 may be coupled to the sensor 30 for treating or for processing the signals received in the sensor 30. The signal received in the sensor 30 may be very week, for example, may be ranged from 0.5 to 1.0 mv. As shown in FIG. 7, an electric circuit or facility may be provided and received in the control device 40 and includes a filter 31 coupled to the sensor 30 for filtering and removing the unwanted signals, a comparator 32 coupled to the filter 31, a sample signal 33 supplied to the comparator 32 which may compare the signals from the sensor 30 and the filter 31 and from the sample signal 33, and an amplifier 34 coupled to the comparator 32 for amplifying the compared signal from the comparator 32. The control device 40 may further include a processor unit to receive and to treat the signals from the amplifier 34.

It is to be noted that the trachea 70 of people is normally about 25 mm inside the throat portion or distant or spaced from the sensor 30 for about 25 mm. The signal emitting device 20 that is located or engaged in the esophagus 80 may be spaced from the sensor 30 for about or more than 30 mm. The sensor 30 may be arranged or used to detect the signals within about 25 mm, i.e., the signals in the range of about the trachea 70 of the patient. For example, when the signal emitting device 20 is located or engaged in the trachea 70 of the patient or located in the range of about 25 mm, the sensor 30 may detect the signals from the signal emitting device 20 and may thus detect and locate that the tube 10 is correctly engaged in the trachea of the patient. For such circumstances, the control device 40 may include or provide a lamp 41 and/or an alarm device 42 for generating a light signal or a sound signal in order to indicate that the signal emitting device 20 and thus the tube 10 are correctly engaged in the trachea of the patient. No signals may be detected by the sensor 30 when the signal emitting device 20 is engaged in the esophagus 80. The control device 40 may further includes a displayer or a meter 43 or the like and may include a processor unit to treat the signals and/or to calculate the position of the signal emitting device 20 spaced from the sensor 30 and to display the distance of the signal emitting device 20 from the sensor 30 in the displayer or the meter 43.

One or more batteries may be received in the control device 40 to energize the electric facilities which may also be coupled to the other electric power supply in order to be energized and actuated. The signal emitting device 20 and the sensor 30 and the control device 40 may be easily carried by the nurses or the like and may be easily used for emergency uses.

Accordingly, the detecting device for correct endotracheal intubation in accordance with the present invention may be used for easily detecting the correct engagement of the tube into the trachea and for preventing the tube from being engaged into the esophagus of the patients. In addition, the detecting device may be easily carried outdoors and thus may be easily used for emergency or intensive care or other resuscitation purposes.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A detecting device for correct endotracheal intubation comprising:
a tube to be engaged into a trachea of a patient,
a signal emitting device for engaging into the trachea of the patient and for emitting a signal, and
means for sensing the signal from said signal emitting device to detect the correct engagement of said tube in the trachea of the patient.

2. The detecting device for correct endotracheal intubation according to claim 1, wherein said signal emitting device is attached to said tube for engaging into the trachea of the patient.

3. The detecting device for correct endotracheal intubation according to claim 1, wherein said signal emitting device is a magnet for emitting a magnetic signal.

4. The detecting device for correct endotracheal intubation according to claim 1, wherein said sensing means includes a sensor for sensing the signal from said signal emitting device.

5. The detecting device for correct endotracheal intubation according to claim 4, wherein said sensing means further includes means for treating the signal from said sensor.

6. The detecting device for correct endotracheal intubation according to claim 1 further comprising means for indicating the correct engagement of said tube in the trachea of the patient.

7. The detecting device for correct endotracheal intubation according to claim 6, wherein said indicating means includes a lamp for generating a light signal.

8. The detecting device for correct endotracheal intubation according to claim 6, wherein said indicating means includes an alarm device for generating an alarm signal.

9. The detecting device for correct endotracheal intubation according to claim 6, wherein said indicating means includes a meter for indicating a distance between said sensing means and said signal emitting device.
